(19) United States
(12) Patent Application Publication
(10) Pub. No.: US 2005/0119270 A1
(43) Pub. Date: Jun. 2, 2005

(54) Synergistic effect of amlodipine and atorvastatin on aortic endothelial cell nitric oxide release

(76) Inventor: R. Preston Mason, Manchester, MA (US)

Correspondence Address: PERKINS, SMITH & COHEN LLP, ONE BEACON STREET, 30TH FLOOR, BOSTON, MA, 02108, US

(21) Appl. No.: 10/987,271

(22) Filed: Nov. 12, 2004

Related U.S. Application Data
(63) Continuation-in-part of application No. 09/921,479, filed on Aug. 3, 2001, now Pat. No. 6,835,742.
(60) Provisional application No. 60/223,214, filed on Aug. 4, 2000.

(51) Int. Cl. A61K 31/525; A61K 31/401; A61K 31/455

(52) U.S. Cl. 514/251; 514/355; 514/423; 514/458; 514/565; 514/411; 514/651

(57) ABSTRACT

The combination of amlodipine and atorvastatin act to synergistically synthesize NO production. Moreover, the addition of a tertiary compound complements this combination of amlodipine and atorvastatin in NO production.

### CROSS-REFERENCE TO RELATED APPLICATIONS

This continuation-in-part application claims the benefit of and priority to U.S. patent application Ser. No. 09/921,479, filed Aug. 3, 2001 which claims the benefit of and priority to U.S. Provisional Patent Application No. 60/223,214, filed on Aug. 4, 2000.

### FIELD OF THE INVENTION

This invention relates to the effect of amlodipine and atorvastatin, alone, or in combination with one another, or with one another plus a tertiary agent, on the production and release of nitric oxide (NO) from endothelial cells.

### BACKGROUND OF THE INVENTION

Coronary artery disease (CAD) is the leading cause of mortality in the developed world, and is associated with substantial morbidity as well. Typically, the patient with CAD has several concomitant conditions, including hypertension, diabetes, and dyslipidemia, increasing overall risk for poor outcomes and complicating treatment. A therapeutic goal for the treatment of CAD is the development of drugs that can simultaneously target multiple underlying disease processes that contribute to atherosclerosis, thereby altering the course of the disease. Therefore, CAD therapy may have increased positive outcomes if the use of an antihypertensive agent and HMG-CoA reductase inhibitor was combined in a single delivery system.

Free cholesterol is an important structural component of the cell plasma membrane that modulates packing of phospholipid molecules, thus regulating lipid bilayer dynamics and structure. The cholesterol molecule is oriented in the membrane such that the long-axis lies parallel to the phospholipid acyl chains, increasing order in the upper acyl chain region of the membrane while decreasing packing constraints at the terminal methyl groups. During atherogenesis, however, increasing levels of cellular cholesterol lead to its abnormal deposition in the vessel wall and the formation of cholesterol crystals.

In animal models of atherosclerosis, it has been demonstrated that the cholesterol content of membranes associated with vascular smooth muscle and macrophage foam cells becomes elevated, resulting in the formation of discrete domains. These highly organized cholesterol structures, characterized by a unit cell periodicity of 34.0 Å, appear to serve as nucleating sites for the formation of extracellular crystals. These domains have been previously described in model membrane systems. A recent study from our laboratory showed that cultured mouse peritoneal macrophage foam cells produced free cholesterol crystals that extend from intracellular membrane sites with various morphologies that include plates, needles and helices. With the use of x-ray diffraction approaches, the early stages of crystal formation could be identified in isolated membranes from these cells. Preventing crystal formation is an important goal as cholesterol in this state is practically inert and does not respond well to pharmacologic interventions that promote lesion regression.

In addition, the normal production of NO by the endothelium is critical for maintaining vascular function. During atherosclerosis, however, endothelial dysfunction effects a significant reduction in NO production, resulting in: 1) increased monocyte and LDL infiltration, 2) loss of smooth muscle cell function and abnormal proliferation, 3) increased oxidative stress, and 4) increased platelet aggregation. Pharmacologic interventions that restore endothelial function and NO metabolism have demonstrated benefit in the treatment of various cardiovascular disorders, including coronary artery disease.

A pharmaceutical composition that treats both hypertension and hyperlipidemia would have several benefits. For example, the multiple risk factors for arterial and related heart disease that are often present in an individual patient could be targeted simultaneously. Additionally, the ease of taking one combined dosage could significantly enhance patient compliance with therapeutic regimens.

Therefore, it is an object of this invention to provide a combination therapy that will treat the multiple pathological processes involved in arterial and related heart disease. These include, but are not limited to, hypertension and hyperlipidemia. It is also an object of this invention to develop useful and convenient dosage levels and forms of such a combination therapeutic. Preferably, this pharmaceutical composition would have synergistic effects on these hallmarks of arterial and related heart disease, such that the individual effects of the components of this composition would be enhanced by their combination.

Thus, this invention encompasses a therapeutic goal for the treatment of CAD that entails the development of drugs that can simultaneously target multiple underlying disease processes that contribute to atherosclerosis, thereby altering the course of the disease. Therefore, using this invention, CAD therapy may have increased positive outcomes if the use of an antihypertensive agent and HMG-CoA reductase inhibitor was combined in a single delivery system.

The clinical manifestations of atherosclerosis, including coronary artery disease and stroke, are the leading cause of death and disability in the United States. Atherosclerosis, in turn, is causally linked to an impairment of endothelium-dependent relaxations, characterized by reduced bioavailability of nitric oxide (NO) produced from endothelial NO synthase (eNOS). Indeed, the major risk factors for atherosclerosis such as hyperlipidemia, diabetes, obesity, heart failure, hypertension, and smoking are all associated with impaired endothelium-dependent relaxation (EDR). Although the underlying mechanisms of the reduced EDR are multifactorial, its most important cause is a disruption of the nitric oxide (NO) pathway. Thus, agents that enhance and restore the normal production of NO would represent an important new development in the treatment of atherosclerosis, and ultimately, cardiovascular disease. We have recently discovered that the combination of amlodipine and atorvastatin synergistically affects NO bioavailability. There is a current desire to combine these agents with a third agent that would further enhance NO bioavailability.

### SUMMARY OF THE INVENTION

This invention relates to the effect of amlodipine and atorvastatin, alone, or in combination with one another, or with one another plus a tertiary agent, on the production and release of nitric oxide (NO) from endothelial cells.

One embodiment of the present invention is directed to a pharmaceutical composition for enhancing NO production comprising therapeutically effective amounts of amlodipine, atorvastatin and a NO enhancing tertiary compound. In one aspect of this embodiment, the atorvastatin can be either atorvastatin itself or its hydroxylated metabolite. In yet another aspect, the NO enhancing tertiary agent can be, for example, L-arginine, tetrahydrobiopterin, an ACE-inhibitor, an antioxidant, a β-blocker, an angiotensin II type 1-receptor antagonist and alike.

In yet another embodiment, a method of synergistically increasing nitric oxide production by endothelial cells comprising administering a therapeutically effective amount of a combination of amlodipine, an atorvastatin compound, and an NO enhancing tertiary agent is described.

In still another embodiment, a method of treating arterial and related heart disease comprising administering a therapeutically effective amount of a combination of amlodipine, an atorvastatin compound, and an NO enhancing tertiary agent is described.

Another embodiment of the present invention is directed to a method of lowering blood pressure and systemic lipid concentrations comprising administering a therapeutically effective amount of a combination of amlodipine, an atorvastatin compound, and an NO enhancing tertiary agent.

### BRIEF DESCRIPTION OF THE DRAWINGS

FIG. 1 shows the X-ray diffraction pattern and corresponding molecular model for cholesterol-enriched membrane bilayer. Diffraction peaks corresponding to sterol-rich and -poor domains can be clearly distinguished at 87% relative humidity at 20° C. The peaks labeled 1′ and 2′ correspond to the sterol-rich domain (d=34.0 Å) while the surrounding sterol-poor area of the membrane had a d-space value of 60.7 Å, corresponding to peaks labeled 1, 2 and 4. The corresponding molecular model demonstrates cholesterol bilayer domain with a dimension of 34.0 Å (each individual cholesterol monohydrate molecule is 17.0 Å) that is highlighted by the shaded region of the figure.

FIG. 2 shows the differential effects of temperature (FIG. 2A) and relative humidity (FIG. 2B) on the molecular dimensions of cholesterol monohydrate domains versus surrounding sterol-poor membrane regions for samples containing verapamil. The membrane width, as measured in A units by x-ray diffraction analysis, represents the distance from the center of one membrane to the next, including surface hydration. In FIG. 2A, the effect of temperature on membrane width was evaluated at a constant 93% relative humidity while in FIG. 2B the effect of relative humidity was measured at a constant temperature of 20° C. These data demonstrate that the structure of the cholesterol monohydrate crystalline domains (34.0 Å) are unaffected by changes in temperature or humidity, as compared to the surrounding sterol-poor region of the membrane.

FIG. 3 shows the X-ray diffraction pattern from oriented membrane lipid bilayers containing elevated levels of cholesterol (1.1:1 and 1.2:1 cholesterol to phospholipid mole ratios) prepared in the absence or presence of the AML/AT combination at 5° C. At a 1.1:1 cholesterol to phospholipid mole ratio, peaks labeled 1, 2 and 4 correspond to d-space values of 54.2 Å and 53.0 Å, respectively, for the control and drug-containing samples. At a 1.2:1 cholesterol to phospholipid mole ratio, peaks labeled 1 and 2 corresponded to d-space values of 55.5 Å and 53.5 Å, respectively, for the control and drug-containing samples. This figure demonstrates that at a low concentration (30 nM), the combination of AML and AT completely blocked the aggregation of cholesterol into discrete cholesterol domains.

FIG. 4 shows the X-ray diffraction patterns from oriented membrane lipid bilayers containing elevated levels of cholesterol (1.2:1 cholesterol to phospholipid mole ratio) prepared in the absence or presence of AML alone, AT alone, AML/AT combination, AT/nifedipine combination, and AML/lovastatin combination at 5° C. The peaks labeled 1, 2 and 4 correspond to the sterol-poor region of the membrane while peaks labeled 1′ and 2′ correspond to the structure of cholesterol monohydrate domains within the membrane (34.0 Å). The dimensions of the surrounding sterol-poor regions were as follows: control (55.5 Å), AML alone (57.8 Å), AT alone (56.8 Å), AML/AT (53.5 Å), AT/nifedipine (56.5 Å) and AML/lovastatin (54.4 Å). These experiments demonstrated that the ability of the AML/AT combination to interfere with membrane cholesterol domain formation could not be reproduced by the drugs separately or other CCB/statin combinations.

FIG. 5 shows the X-ray diffraction patterns from oriented membrane lipid bilayers containing elevated levels of cholesterol (1.1:1 cholesterol to phospholipid mole ratio) prepared in the absence or presence of AML alone, AT alone, and AML/AT combination at 5° C. The peaks labeled 1, 2 and 4 correspond to the sterol-poor region of the membrane while peaks labeled 1′ and 2′ correspond to the structure of cholesterol monohydrate domains within the membrane (34.0 Å). The dimensions of the surrounding sterol-poor regions were as follows: control (52.4 Å), AML alone (54.4 Å), AT alone (55.8 Å), and AML/AT (53.9 Å). These experiments demonstrated that the AML/AT combination was able to interfere with membrane cholesterol domain formation in a manner that could not be reproduced by the drugs separately.

FIG. 6 shows the dose response curves for NO release stimulated by amlodipine, atorvastatin (Compound T), and a mixture of amlodipine with varying concentrations of atorvastatin (Compound T).

FIG. 7 depicts the effect of amlodipine, atorvastatin either alone or in combination on NO synthesis.

### DETAILED DESCRIPTION OF THE INVENTION

This invention relates to the effect of amlodipine and atorvastatin, alone, or in combination with one another, or with one another plus a tertiary agent, on the production and release of nitric oxide (NO) from endothelial cells.

One embodiment of the present invention is directed to a pharmaceutical composition for enhancing NO production comprising therapeutically effective amounts of amlodipine, atorvastatin and a NO enhancing tertiary compound. In one aspect of this embodiment, the atorvastatin can be either atorvastatin itself or its hydroxylated metabolite. In yet another aspect, the NO enhancing tertiary agent can be, for example, L-arginine, tetrahydrobiopterin, an ACE-inhibitor, an antioxidant, a β-blocker, an angiotensin II type 1-receptor antagonist and alike.

Studies were conducted to examine the effect of combining amlodipine and atorvastatin. The protocol and results are setforth below.

Preparation of reconstituted membrane samples. Porcine cardiac phospholipid dissolved in HPLC-grade chloroform (10.0 mg/ml) was obtained from Avanti Polar Lipids Inc. (Alabaster, Ala.) and stored at −80° C. The fatty acid composition of the phosphatidylcholine lipids was determined by gas-liquid chromatographic analysis. The overall ratio of saturated to unsaturated fatty acids was 0.8:1, with the primary constituents being 18:2 linoleic acid (30%), 16:0 palmitic acid (22%), 18:1 oleic acid (13%), and 20:4 arachidonic acid (11%). Cholesterol powder was also purchased from Avanti Polar Lipids Inc. Amlodipine besylate (AML) was obtained from Pfizer Central Research (Groton, Conn.) while atorvastatin calcium (AT) was provided by Parke Davis (Ann Arbor, Mich.).

The effects of the drugs on membrane cholesterol organization and structure were assessed in well-defined lipid vesicles containing equimolar levels of cholesterol and phospholipid. This reconstituted membrane system was used for the following reasons: 1) this system reproduces changes in membrane structure observed in cholesterol-enriched, atherosclerotic macrophage and smooth muscle cell membranes, 2) the membrane preparation does not contain calcium channels, and 3) these samples can be prepared in a highly reproducible fashion. Lipid vesicles were formed from phospholipid and cholesterol dissolved in chloroform at a fixed molar ratio and added to individual glass 13×100-mm test tubes. The chloroform solvent was removed by shell-drying under a steady stream of N₂ gas. Residual solvent was removed under vacuum while the samples were shielded from light. Membrane vesicles were produced for diffraction analysis by rapidly mixing the dried lipids at room temperature following addition of buffered saline (0.5 mmol/L HEPES and 154.0 mmol/L NaCl, pH, 7.2). The final phospholipid concentration was 5.0 mg/mL. Membrane samples were oriented for diffraction analysis by centrifugation and then placed in hermetically sealed canisters that controlled temperature and relative humidity, as previously described.

Small angle x-ray diffraction analysis. Small-angle x-ray diffraction approaches were used to directly examine the effects of the various drugs on the organization of cholesterol in the membrane. X-ray diffraction experiments were conducted by aligning the samples at grazing incidence with respect to a collimated, nickel-filtered monochromatic x-ray source (CuK_{α}=1.54 Å) produced by a high-brilliance rotating anode microfocus generator (Rigaku Rotaflex RU-200, Danvers, Mass.). The diffraction data were collected on a one-dimensional, position-sensitive electronic detector (Innovative Technologies, Newburyport, Mass.) placed at a distance of 150 mm from the sample. In addition to direct calibration of the detector system, cholesterol monohydrate crystals were used to verify the calibration, as previously described. The unit cell periodicity, or d-space, of the membrane lipid bilayer is the measured distance from the center between one bilayer to the next, including surface hydration, and calculated from Bragg's Law.

NO release measurements. All measurements presented were recorded in vitro. NO release was measured directly from a single endothelial cell in the rabbit aorta. Measurements were done in Hank's balance solution at 37° C. A porphyrinic sensor (diameter 0.2±0.1 μm) was placed near the surface (10±5 μm) of the endothelial cells using a computer controlled micromanipulator. The sensor operated with a three-electrode system [sensor working electrode, platinum wire (0.1 mm) counter electrode, and saturated calomel electrode (SCE—reference electrode)]. The three electrodes were connected to a potentiostat/galvanostat PAR273. Data were acquired with the use of an IBM computer with custom software. The current proportional to NO concentration was measured by porphyrinic sensor, which operated in amperometric mode at constant potential of 0.63 V vs. SCE.

The release of NO was initiated by the injection of potential agonists of endothelial NO synthase (eNOS) using a temtoinjector placed in the controlled distance from the endothelial cell. Two different agonists were tested: amlodipine and atorvastatin. The different concentrations of these two compounds applied simultaneously were also tested.

Atherosclerotic-like membranes have distinct crystalline-like sterol domains: Membrane sterol-rich domains may represent an important nucleating site for free cholesterol crystal formation, an important feature of the unstable plaque. The separate and combined effects of AML and AT on cholesterol monohydrate formation in membranes reconstituted from native phospholipids isolated from cardiac tissue was evaluated. Phospholipid composed of heterogeneous acyl chains was used for these analyses. This membrane system reproducibly formed discrete sterol-rich domains at levels previously observed in atherosclerosis studies under similar experimental conditions.

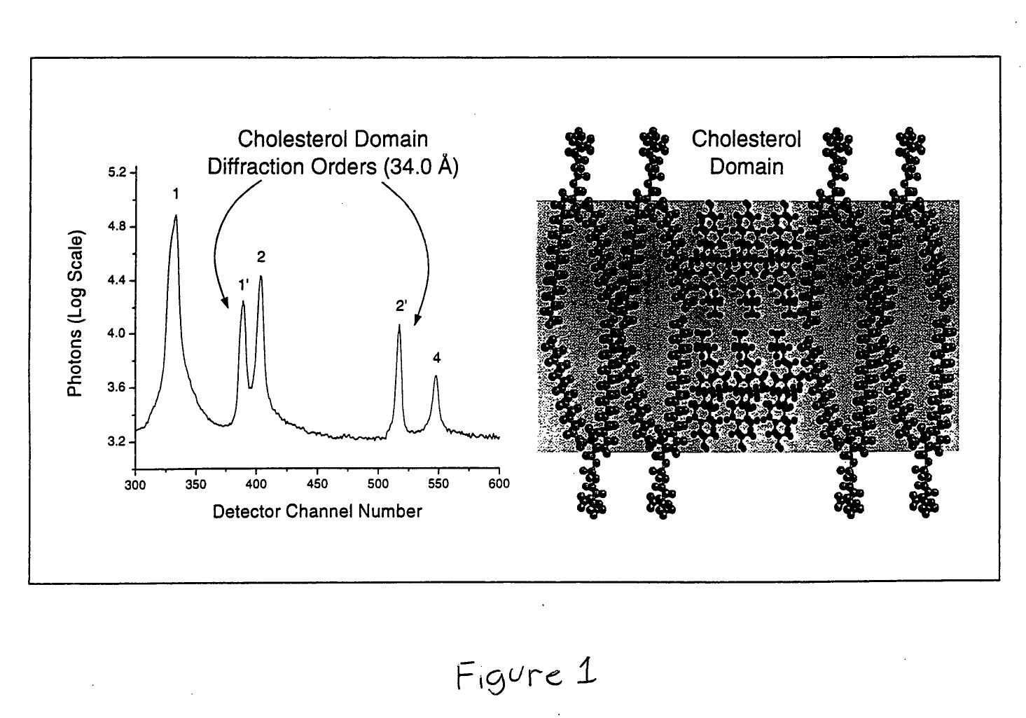

X-ray diffraction analysis of oriented, cholesterol-enriched membranes produced strong, reproducible diffraction orders that correspond to structurally distinct sterol-rich and -poor membrane regions. The d-space measurement refers to the average distance from the center of one membrane bilayer to the next, including surface hydration. The d-space of the sterol-rich region was 34.0 Å, indicative of a cholesterol bilayer structure as a single cholesterol monohydrate molecule has a long axis of 17 Å (FIG. 1). The surrounding sterol-poor regions, meanwhile, had an average width of 65.9 Å at 20° C. and 93% relative humidity. The much larger width (>90%) of the sterol-poor domains is attributed to the abundance of phospholipid in the surrounding membrane region. The cholesterol domains were invariably present over a wide range of temperatures (5-37° C.) and relative humidity levels (74-93%), consistent with previous x-ray diffraction analyses on atherosclerotic-like membrane samples.

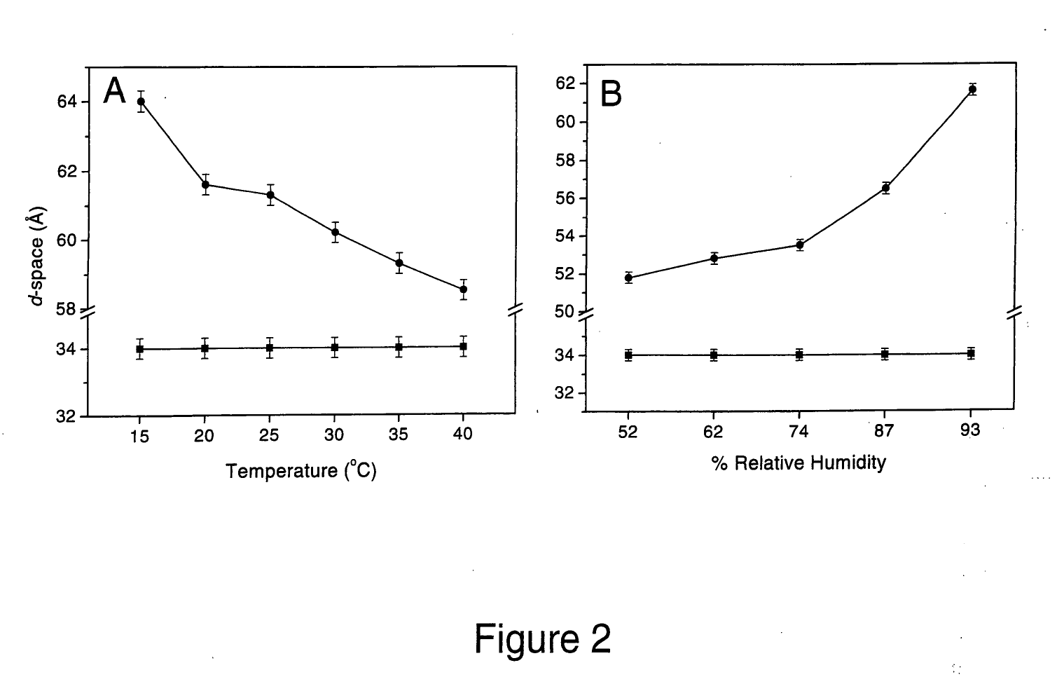

In FIG. 1, diffraction peaks corresponding to the sterol-rich and -poor domains can be clearly distinguished at 20° C. The peaks labeled 1′ and 2′ correspond to the sterol-rich domain (d=34.0 Å) while the surrounding sterol-poor area of the membrane had a d-space value of 60.7 Å, corresponding to peaks labeled 1, 2 and 4. The peaks that describe the cholesterol monohydrate phase are very sharp, as expected for a crystalline-like structure. In every sample that was evaluated, it was observed that the dimensions of the sterol-poor region of the membrane was modulated by temperature and relative humidity due to its heterogeneous chemical composition and the dynamic mobility of the phospholipid-cholesterol binary mixture. At 93% relative humidity, for example, the d-space of the sterol-poor region decreased by 5.5 Å (9%) as sample temperature was increased from 15° C. (64 Å) to 40° C. (58.5 Å), consistent with increased trans-gauche isomerizations (FIG. 2). Over this same temperature range, however, the cholesterol monohydrate phase remained unchanged at 34.0 Å, as expected for a crystalline-like structure. In addition, the highly reproducible 34.0 Å structure was unaffected by large changes in relative humidity (52 to 93%) at 20° C. while the sterol-poor region changed by 19% or 10 Å (52 to 62 Å) over this same range.

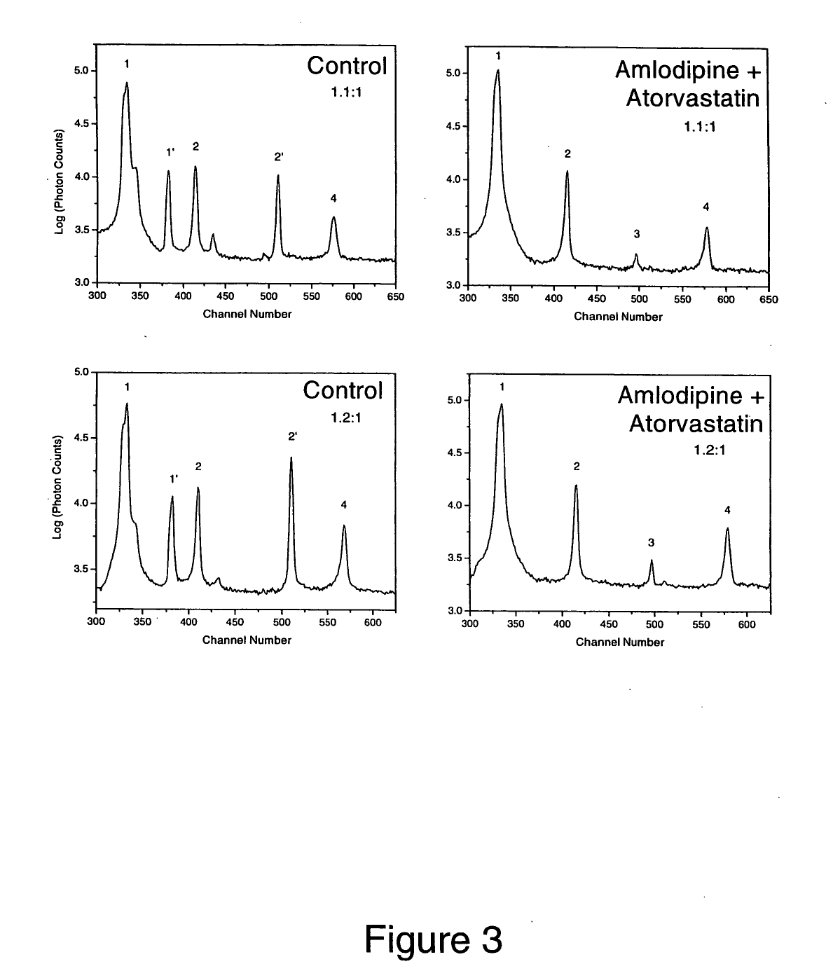

Synergistic inhibition of sterol domain formation with amlodipine and atorvastatin: The addition of both AML and AT to cholesterol-enriched membrane samples prevented sterol domain formation in a synergistic fashion. At an aqueous buffer concentration of 30 nM, the combination of AML and AT completely blocked the formation of cholesterol domains in membrane samples containing cholesterol and phospholipid at 1.1:1 and 1.2:1 cholesterol:phospholipid mole ratios. In the presence of the two drugs, only peaks corresponding to the phospholipid bilayer could be observed under a variety of experimental conditions, as compared to control (FIG. 3). At a 1.1:1 mole ratio, the d-space values for the control and drug combination-containing samples were 54.2 and 53.9 Å, respectively, at 74% relative humidity and 5° C. At a 1.2:1 mole ratio, the d-space values for the control and drug combination-containing samples were 55.5 and 53.5 Å, respectively, at 74% relative humidity and 5° C.

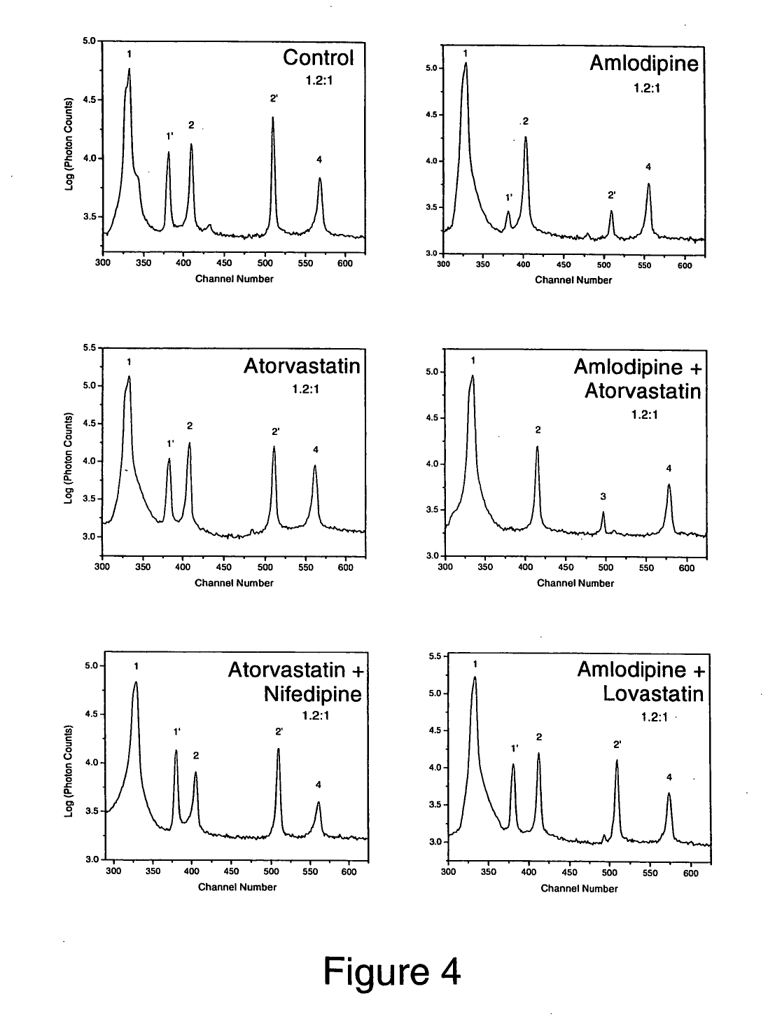

When AML or AT were added separately to the membrane samples, cholesterol domains could be clearly detected under identical conditions with small angle x-ray diffraction approaches. Moreover, the combination of AML and AT with other drugs had no inhibitory effect on cholesterol crystal formation. Both the combination of AML with the HMG-CoA reductase inhibitor lovastatin and the combination of AT with the CCB nifedipine failed to interfere with cholesterol domain formation, as compared to control samples (FIG. 4). Cholesterol domains were very prominent in these samples with a unit cell periodicity of 34.0 Å. These discrete structures coexist with the surrounding sterol-poor region of the membrane. At 5° C. and 74% relative humidity, the surrounding sterol-poor region of the membrane samples had the following d-space values: control (55.5 Å), AML/lovastatin (54.4 Å), and AT/nifedipine (56.5 Å). Finally, when AML and AT were added separately to the cholesterol-enriched membrane samples, they did not interfere with domain formation.

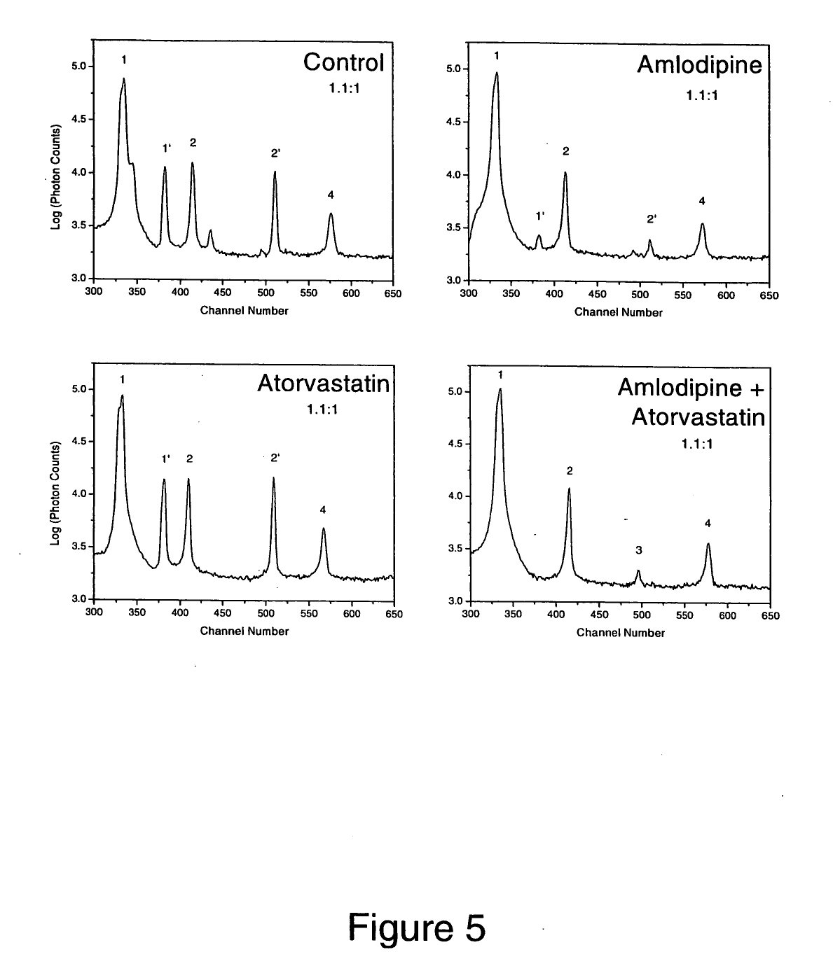

The synergistic effect of AML and AT on cholesterol domain formation was also observed at a lower concentration of cholesterol. At a cholesterol to phospholipid mole ratio of 1.1:1, the drug combination effectively interfered with cholesterol crystallization within the membrane samples (FIG. 5). By contrast, when used separately, the drugs had no effect on domain formation, even at this lower level of membrane cholesterol. At 5° C. and 74% relative humidity, the surrounding sterol-poor region of the membrane samples had the following d-space values: control (55.5 Å), AML alone (54.4 Å), AT alone (55.8 Å), and AML/AT (53.9 Å).

An explanation for the synergistic effect of AML and AT on the organization of cholesterol may be their chemical properties. AML has very high lipophilicity as compared to other CCBs and a formal positive charge at physiologic pH. An electrostatic interaction between AML and AT as well as the phospholipid headgroup region of the membrane contributes to the high affinity of this agent for the lipid bilayer. Moreover, the charged amino-ethoxy function of AML directs the drug to a region of the membrane that overlaps the steroid nucleus of cholesterol molecules, an effect that may directly lead to a disruption in the self-association of cholesterol molecules in the membrane. Likewise, it has been observed that AT partitions to a similar location in the membrane as AML.

The key finding was the observation that the combination of AML and AT inhibited the formation of separate cholesterol domains in atherosclerotic-like membranes in a synergistic fashion. This biophysical effect of the drug combination was directly characterized with small angle x-ray diffraction approaches using lipid membranes enriched with cholesterol. As cholesterol aggregates within the membrane may serve as nucleating sites for extracellular free cholesterol crystal formation in the vessel wall, the ability of the AML/AT combination to block such sterol domain formation indicates a novel antiatherosclerotic mechanism of action. This observed effect appears to be distinct for these drugs as other combinations failed to reproduce this change in the aggregation properties of free cholesterol.

In atherosclerosis, the incidence of lesion rupture and thrombosis is affected by the lipid composition of the atherosclerotic plaque. The lipid component of atherosclerotic lesions consists primarily of cholesterol and phospholipid, with lesser amounts of fatty acid and triacylglycerol. Over time, cholesterol forms crystalline structures in the human atheroma, an event that contributes to overall lesion mass and plaque instability. Once crystallized, cholesterol within the lesion is essentially inert and cannot be effectively removed by lipoprotein acceptors in the plasma. By contrast, non-crystallized cholesterol associated with foam cell membranes or intracellular stores can be depleted by plasma HDL and pharmacological interventions, leading to lesion regression.

Recent reports indicate that the cellular membrane is a cellular site for free cholesterol accumulation, leading to discrete sterol-rich domains and eventually crystal. In macrophage foam cells, for example, a critical mass of cholesterol is achieved following lipoprotein (native or oxidized) uptake and/or phagocytosis of lipid released from neighboring necrotic foam cells. Ultimately, a nucleating event will occur at a critical concentration of cholesterol enrichment, leading to cholesterol domain development within the membrane. By interfering with the formation of highly organized cholesterol aggregates within the membrane, the combination of AML and AT may significantly slow or even prevent subsequent crystal development in the vessel wall, and thereby block the progression of an otherwise irreversible step in atherosclerosis. Moreover, these agents may work synergistically with HDL and lipid-lowering therapy in reducing the accumulation of cholesterol crystals in the wall of the diseased artery by maintaining cholesterol in a non-crystalline or dynamic state in cellular membranes.

The mechanism by which AML and AT interfere with the aggregation of cholesterol into discrete domains may be related to its their molecular membrane interactions. At physiologic pH, more than 90% of the amino ethoxy function associated with the #2 position of the dihydropyridine ring of AML is in the charged state. This positive charge contributes to specific electrostatic interactions of AML with phosphate groups associated with the phospholipid bilayer surface. The results of previous small-angle x-ray diffraction, differential scanning calorimetry and nuclear magnetic resonance analyses support a molecular model that places the charged amino function of AML near oppositely charged groups in the phospholipid headgroup region. Simultaneously, the hydrophobic portion of the dihydropyridine molecule is buried within the membrane hydrocarbon core, adjacent to the headgroup region. These biophysical measurements indicate that the time-averaged location of the ring structure for AML overlaps the sterol nucleus of cholesterol in the membrane, where it can then modulate certain biophysical effects of the molecule, and interfere with its self-association. Likewise, small-angle x-ray diffraction approaches demonstrated that AT partitioned to a discrete location in the membrane bilayer.

Thus, this unexpected, synergistic effect can be attributed to the molecular interactions of these compounds with membrane lipid constituents. This finding has important relevance for the treatment of coronary artery disease (CAD) as this disorder is characterized by the abnormal accumulation of free cholesterol into separate, membrane domains (d-space of 34.0 Å). These domains disrupt cellular function and lead to extracellular crystal formation, an important feature of the unstable atherosclerotic plaque. Small angle x-ray diffraction analyses demonstrated, for the first time, that the combination of AML and AT blocked the aggregation of free cholesterol into crystalline-like domains at low, nanomolar concentrations. By contrast, the combination of these agents with other related drugs showed no inhibitory effect on cholesterol crystal formation. These findings indicate that the combination of AML and AT produces a novel anti-atherosclerotic effect by disrupting cholesterol crystal formation in atherosclerotic-like membranes. By disrupting the formation of cholesterol crystals in the vessel wall, the AML/AT combination would reduce plaque instability while facilitating cholesterol efflux to sterol acceptor particles, such as HDL. This new anti-atherosclerotic mechanism of action for the AML/AT combination would complement the separate activities of these agents in the effective treatment of cardiovascular disease.

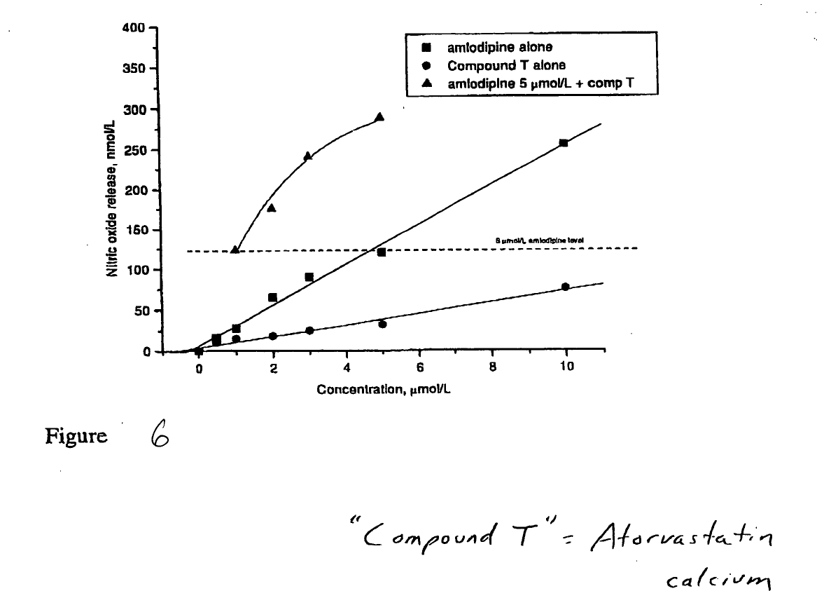

NO Release from Aortic Endothelial Cells: FIG. 6 shows dose response curves for NO release stimulated by amlodipine, atorvastatin, and the mixture of 5 μmol/L of amlodipine and variable concentrations (from 1-5 μmol/L) of atorvastatin. Based on the data depicted in FIG. 6, there is a significant synergistic effect observed after stimulation of NO release from endothelial cells by the combination of amlodipine and atorvastatin over a range of doses.

Therefore, the results of these analyses demonstrated a powerful synergistic effect for the combination of amlodipine and atorvastatin on the inhibition of cholesterol crystal formation and nitric oxide release from rabbit aortic endothelial cells. The results of this study provide compelling scientific support for the combined use of AML and AT in the treatment of cardiovascular disorders. These novel antiatherosclerotic effects of the AML/AT combination complement the separate activities of these agents in the treatment of cardiovascular disease, including CAD.

The present invention describes methods for synergistically increasing nitric oxide (NO) release present in a subject's vasculature by administering an effective amount of amlodipine and atorvastatin metabolite with at least one other NO enhancing tertiary agent that enhances NO bioavailability from endothelial cells.

Nitric oxide (NO) is produced by the enzymatic conversion of the amino acid L-arginine to L-citrulline by the enzymatic action of an NADPH-dependent NO synthase (NOS). The NOS enzyme requires Ca²⁺/calmodulin, FAD, FMN, and tetrahydrobiopterin (BH4) as cofactors (Moncada and Higgs, 1993, N. Engl J Med. 329:2002-2012; Nathan and Xie, 1994, J Biol. Chem. 269:13725-28, the entire teachings of which are incorporated herein by reference). In the blood vessels, NO is produced from the endothelium by constitutive expression of the endothelial isoform of NOS (eNOS), which is activated by mechanical stress such as blood shear-stress and stimulation with agonists such as bradykinin and acetylcholine. NO has a variety of functions, but its action as the endothelium-derived relaxing factor (EDRF) is the most important for the maintenance of vascular homeostasis (Moncada and Higgs, 1993).

An impairment of endothelium-dependent relaxations (EDR) is present in atherosclerotic vessels even before vascular structural changes occur and represents the reduced eNOS-derived NO bioavailability. Endothelial dysfunction as characterized by an impairment of EDR, and thereby reduced eNOS-derived NO bioactivity, is the critical step for atherogenesis. Among various mechanisms responsible for the impaired EDR, the increased NO breakdown by superoxide is important, and there is augmented production of superoxide in atherosclerotic vessels. Under certain circumstances, eNOS becomes dysfunctional and produces superoxide rather than NO. The pathophysiological role of dysfunctional eNOS has attracted attentions in vascular disorders, including atherosclerosis.

As previously mentioned, under normal conditions, NO is generated by vascular endothelium nitric oxide synthase (eNOS) in response to activation of mechanochemical receptors associated with increased vascular flow and natural agonists such as acetylcholine, bradykinin and substance P. Endothelial dysfunction, including loss of normal NO production, is associated with various cardiovascular disorders including atherosclerosis, hypertension, heart failure, and diabetes mellitus (see, Drexler H, Hayoz D, Munzel T, Hornig B, Just H, Brunner H R, Zelis R., Endothelial function in chronic congestive heart failure, Am. J. Cardiol. 1992;69:1596-1601; Gilligan D M, Panza J A, Kilcoyne C M, Waclawiw M S, Casion P R, Quyyumi A A., Contribution of endothelium-derived nitric oxide to exercise-induced vasodilation. Circulation. 1994;90:2853-2858; Panza J A, Quyyumi A A, Brush J E, Epstein S E. Abnormal endothelium-dependent vascular relaxation in patients with essential hypertension. N. Engl. J. Med. 1990;323:22-27; Cardillo C, Kicoyne C M, Quyyumi A A, Cannon R O, Panza J A. Selective defect in nitric oxide synthesis may explain the impaired endothelium-dependent vasodilation in patients with essential hypertension. Circulation. 1998;97:851-856; Drexler H, Hornig B. Endothelial dysfunction in human disease. J. Mol. Cell. Cardiol. 1999;3:51-60, the entire teachings of which are incorporated herein by reference.)

In patients with documented hypertension, decreased NO production results in loss of normal vasodilation. During the development of heart failure, endothelial dysfunction results in maladaptive changes in the peripheral vasculature and skeletal muscle, leading to symptoms of exercise intolerance (Drexler H, Hayoz D, Munzel T, Hornig B, Just H, Brunner H R, Zelis R. Endothelial function in chronic congestive heart failure. Am. J. Cardiol. 1992;69:1596-1601; Gilligan D M, Panza J A, Kilcoyne C M, Waclawiw M S, Casion P R, Quyyumi A A. Contribution of endothelium-derived nitric oxide to exercise-induced vasodilation. Circulation. 1994;90:2853-2858, the entire teachings of which are incorporated herein by reference).

Production of NO appears to be an essential activity of the endothelium for maintaining a smooth, nonthrombogenic surface. During atherosclerosis, however, a deficiency in NO synthesis has adverse consequences on vascular hemodynamics and inflammation (Libby P. Changing concepts in atherogenesis. J. Intern. Med. 2000;247:349-358; Ross R. Atherosclerosis—An inflammatory disease. N. Engl. J. Med. 1999;340:115-126, the entire teachings of which are incorporated herein by reference). These deleterious effects include: 1) increased free radical damage, 2) platelet aggregation, 3) increased hyperadhesiveness of leukocytes, 4) enhanced vasoconstriction, and 5) increased production of the vasoconstrictor, endothelin. Thus, a deficiency in NO availability could be a key early event that promotes atherogenesis in the human vasculature.

Pharmacologic agents that enhance NO synthesis have favorable effects on patients with hypertension and atherosclerotic disease (i.e., coronary artery disease) by increasing constitutive levels of eNOS (Wiemer G, Linz W, Hatrik S, Scholkens B A, Malinski T. Angiotensin-converting enzyme inhibition alters nitric oxide and superoxide release in normotensive and hypertensive rats. Hypertension. 1997;30:1183-1190; Treasure C B, Klein J L, Weintraub W S, Talley J D, Stillabower M E, Kosinski A S, Zhang J, Boccuzzi S J, Cedarholm J C, Alexander R W. Beneficial effects of cholesterol-lowering therapy on the coronary endothelium in patients with coronary artery disease. N. Engl. J. Med. 1995;332:481-487, the entire teachings of which are incorporated herein by reference). Surprisingly, the combination of amlodipine and atorvastatin enhances NO production from human endothelial cells in a highly synergistic fashion. This finding has broad implications for the use of these agents in the treatment of cardiovascular diseases.

In one aspect, methods for increasing nitric oxide (NO) release present in a subject's vasculature by administering an effective amount of amlodipine and atorvastatin metabolite with at least one other agent that enhances NO bioavailability from endothelial cells are described. Examples of suitable enhancing NO tertiary agents include, but are not limited to, L-arginine (substrate for NOS), tetrahydrobiopterin (BH4, a co-factor of NOS), ACE-inhibitors (ramipril, enalapril, quinapril), antioxidants (e.g., vitamin E, probucol, vitamin C), β-blockers (nebivolol, carvedilol, metoprolol) and angiotensin II type 1 (AT1)-receptor antagonists (irbesartan, candesartan, valsartan, losartan).

One aspect of the present embodiment is directed toward administering an effective amount of amlodipine/atorvastatin metabolite with a peroxisome proliferator activated receptor (PPARγ) agonists (e.g., rosiglitazone). These agents are used for the treatment of diabetes by enhancing sensitivity of cells to insulin. However, these agents have shown additional vascular benefits beyond genomic regulation, resulting in improved blood pressure and vessel function consistent with endothelial improvement (Ryan et al. 2004 Hypertension, 43:661-666, the entire teaching of which is incorporated herein by reference).

A particular aspect of the present embodiment is directed toward a method for treating a subject that has an endothelial cell dysfunction. The endothelial cell dysfunction causes or contributes to one or more cardiovascular disorders. In a further aspect, the cardiovascular disorder is selected from the group consisting of atherosclerosis, hypertension, dyslipidemia, diabetes mellitus, heart failure, obesity, smoking and renal failure. These subjects can be administered an effective amount of a combination of amlodipine, atorvastatin, and a third agent, such as those described above.

Any of the identified compounds of the present invention can be administered to a subject, including a human, by itself, or in pharmaceutical compositions where it is mixed with suitable carriers or excipients at doses therapeutically effective to prevent, treat or ameliorate a variety of disorders, including those characterized by that outlined herein. A therapeutically effective dose further refers to that amount of the compound sufficient result in the prevention or amelioration of symptoms associated with such disorders. Techniques for formulation and administration of the compounds of the instant invention may be found in Goodman and Gilman's The Pharmacological Basis of Therapeutics, Pergamon Press, latest edition.

The compounds of the present invention can be targeted to specific sites by direct injection into those sites. Compounds designed for use in the central nervous system should be able to cross the blood-brain barrier or be suitable for administration by localized injection.

Pharmaceutical compositions suitable for use in the present invention include compositions wherein the active ingredients are contained in an effective amount to achieve its intended purpose. More specifically, a therapeutically effective amount means an amount effective to prevent development of or alleviate the existing symptoms and underlying pathology of the subject being treating. Determination of the effective amounts is well within the capability of those skilled in the art.

For any compound used in the methods of the present invention, the therapeutically effective dose can be estimated initially from cell culture assays. For example, a dose can be formulated in animal models to achieve a circulating concentration range that includes the IC₅₀ (the dose where 50% of the cells show the desired effects) as determined in cell culture. Such information can be used to more accurately determine useful doses in humans.

A therapeutically effective dose refers to that amount of the compound that results in the attenuation of symptoms or a prolongation of survival in a subject. Toxicity and therapeutic efficacy of such compounds can be determined by standard pharmaceutical procedures in cell cultures or experimental animals, e.g., for determining the LD₅₀ (the dose lethal to 50% of a given population) and the ED₅₀ (the dose therapeutically effective in 50% of a given population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of a patient's condition. Dosage amount and interval can be adjusted individually to provide plasma levels of the active moiety which are sufficient to maintain the desired effects.

In case of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The pharmaceutical compositions of the present invention can be manufactured in a manner that is itself known, e.g., by means of conventional mixing, dissolving, granulating, levigating, emulsifying, encapsulating, entrapping or lyophilizing processes.

Pharmaceutical compositions for use in accordance with the present invention thus can be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention can be formulated in aqueous solutions, preferably in physiologically compatible buffers such as Hank's solution, Ringer's solution, or physiological saline buffer. For transmucosal administration, penetrants appropriate to the barriers to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinyl-pyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage for, e.g., in ampoules or in multidose containers, with an added preservatives. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspension. Suitable lipohilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations previously described, the compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

A pharmaceutical carrier for the hydrophobic compounds of the invention is a co-solvent system comprising benzyl alcohol, a non-polar surfactant, a water-miscible organic polymer, and an aqueous phase. Naturally, the proportions of a co-solvent system can be varied considerably without destroying its solubility and toxicity characteristics. Furthermore, the identity of the co-solvent components can be varied.

Altenatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds can be delivered using a sustained-release system, such as semipermeable matrices of solid hydrophobic polymers containing the therapeutic agent. Various of sustained-release materials have been established and are well known to those skilled in the art. Sustained-release capsules can, depending on their chemical nature, release the compounds for a few weeks up to over 100 days. Depending on the chemical nature and the biological stability of the therapeutic reagent, additional strategies for protein stabilization can be employed.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration can, e.g., include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, e.g., via injection of the compound directly into an affected area, often in a depot or sustained release formulation.

Furthermore, one can administer the compound in a targeted drug delivery system, e.g., in a liposome coated with an antibody specific for affected cells. The liposomes will be targeted to and taken up selectively by the cells.

The compositions can, if desired, be presented in a pack or dispenser device which can contain one or more unit dosage forms containing the active ingredient. The pack can, e.g., comprise metal or plastic foil, such as a blister pack. The pack or dispenser device can be accompanied by instruction for administration. Compositions comprising a compound of the invention formulated in a compatible pharmaceutical carrier can also be prepared, placed in an appropriate container, and labeled for treatment of an indicated condition. Suitable conditions indicated on the label can include treatment of a disease such as described herein.

### EXAMPLE

The following is an experiment that demonstrates the combination of amlodipine and atorvastatin stimulated nitric oxide production from human endothelial cells in a synergistic fashion as compared to control. These data demonstrate a synergistic effect of this unique combination of compounds in treating the disease state of atherosclerosis, which is the underlying disease process for various cardiovascular disorders, including coronary artery disease and heart failure. As discussed above, a deficiency in nitric oxide production is associated with endothelial dysfunction, a major cause of hypertension and atherosclerosis.

The protocol employed is set forth below.

Nanosensor Measurements of Nitric Oxide:

1. Nanosensors were prepared from carbon fibers. The size of the tip of carbon fiber was reduced from 6 μm to less than 1 μm by temperature controlled burning. The sensors were made sensitive to NO by deposition of electrically conductive polymeric porphyrin and covered with a thin layer of Nafion according to the procedures previously described (Malinski T, Taha Z. Nitric oxide release from a single cell measured in situ by a porphyrinic-based microsensor. Nature. 1992;358:676-678, the entire teaching of which is incorporated herein by reference).

2. Measurements of NO were made in the growth medium solution. The nanosensor was positioned at a distance of about 5±2 μm from the surface of endothelial cell with a help of a motorized computer micromanipulator. The nanosensor operates as a component of a three-electrode system: nanosensor (working electrode), saturated calomel electrode (reference electrode) and platinum wire (counter electrode, 0.5 mm diameter).

The nanosensor operates at a constant potential of 0.68 V versus saturated colomel electrode.

Amperograms (current vs. time curves) were recorded with a Guniry FAS1 Femtostat (Warminster, Pa.).

3. HUVEC cells were obtained from American Type Culture Collection (Manassas, Va.) and grown in Ham's F 12K medium with 2 mM L-glutamine adjusted to contain 1.5 g/L sodium bicarbonate and supplemented with 0.1 mg/ml heparin and 0.03-0.05 mg/mL endothelial cell growth supplement (ECGS)+10% fetal bovine serum. The HUVEC cells were kept in the atmosphere of elevated CO₂ concentration (5%).

4. For the measurements cell wells were transferred to a Faraday cage and, with the help of inverted microscope (Leica Microsystems, Wetzlar, Germany) and micromanipulator, the nanosensor was positioned near the surface of HUVEC. The baseline was stabilized after about 20 seconds.

5. Amlodipine, Atorvastatin or the mixture of the two drugs was injected with the help of a nanoinjector. The NO concentration was measured for about 60 seconds.

6. The nanosensor for NO was calibrated using saturated solution (concentration 1.82 mmol/L verified with the coulometric method).

7. Prepared stock solutions:
A) Amlodipine:
   Weight=51.5 mg, MW=567.1
   Stock Solution: 10 μM in ethanol
   take 5.7 mg and dissolve in 1 mL of ethanol.
B) Atorvastatin:
   Weight=53.6 mg, MW=585.68
   Stock Solution: 10 μM in methanol
   take 5.9 mg and dissolve in 1 mL of methanol.

8. Sample solutions of Amlodipine and Atorvastatin were prepared as follows. Nine separate concentrations of Amlodipine and Atorvastatin were tested: 0.25; 0.75; 1.00; 1.50; 2.00; 2.50; 3.00 and 5.00 μM. The working solutions were prepared by dilution of stock solutions with distilled water.

The Pipetting Scheme was a follows:

A) Amlodipine and Atorvastatin (both μM stock)

TABLE 1
Amlodipine and Atorvastatin (both μM stock)

| Concentration (μM) | Concentration (μM) | μl of Stock | μl of Water |
|---|---|---|---|
| Vial | Final | | |
| 50.0 | 0.25 | 5 | 995 |
| 150.0 | 0.75 | 15 | 985 |
| 200.0 | 1.00 | 20 | 980 |
| 300.0 | 1.50 | 30 | 970 |
| 400.0 | 2.00 | 40 | 960 |
| 500.0 | 2.50 | 50 | 950 |
| 600.0 | 3.00 | 60 | 940 |
| 1000.0 | 5.00 | 100 | 900 |

B) The working solutions had a concentration 200× times higher than required (final) as the cell well volume was 2 mL while the injected volume was 10 μL (200× dilution).

9. The synergistic effect was tested at a constant concentration (5 μM) of Amlodipine (A) and variable concentrations of Atorvastatin (T). The next series of experiments tested this effect at constant ratios of both compounds according to formulations (A:T):

1 μM of A:1 μM of T; 2 μM of A:2 μM of T; 2.5 μM of A:2.5 μM of T; 3.0 μM of A: 3.0 μM of T; 5.0 μM of A:5.0 μM of T.

10. Peak of maximal NO concentration was calculated.

11. Area under current vs. time curve (amperogram) was integrated (coulometry) and amount of NO detected by the nanosensor was calculated.

The following HUVEC samples were analyzed in triplicate at 37° C. The method used was described above.

TABLE 2
Amlodipine

```
  0 μM - Control #1
  0 μM - Control #2
  0 μM - Control #3
0.25 μM - 1 of 3
0.25 μM - 2 of 3
0.25 μM - 3 of 3
0.75 μM - 1 of 3
0.75 μM - 2 of 3
0.75 μM - 3 of 3
  1.0 μM - 1 of 3
  1.0 μM - 2 of 3
  1.0 μM - 3 of 3
  1.5 μM - 1 of 3
  1.5 μM - 2 of 3
  1.5 μM - 3 of 3
  2.0 μM - 1 of 3
  2.0 μM - 2 of 3
  2.0 μM - 3 of 3
  2.5 μM - 1 of 3
  2.5 μM - 2 of 3
  2.5 μM - 3 of 3
  3.0 μM - 1 of 3
  3.0 μM - 2 of 3
  3.0 μM - 3 of 3
  5.0 μM - 1 of 3
  5.0 μM - 2 of 3
  5.0 μM - 3 of 3
```

Atorvastatin

The Atorvastatin data were recorded in a similar manner as Amlodipine data.

TABLE 3
Mixture: Amlodipine (5 μM) + Atorvastatin (varies)
Atorvastatin

```
0.25 μM - 1 of 3
0.25 μM - 2 of 3
0.25 μM - 3 of 3
0.75 μM - 1 of 3
0.75 μM - 2 of 3
0.75 μM - 3 of 3
  1.0 μM - 1 of 3
  1.0 μM - 2 of 3
  1.0 μM - 3 of 3
  1.5 μM - 1 of 3
  1.5 μM - 2 of 3
  1.5 μM - 3 of 3
  2.0 μM - 1 of 3
  2.0 μM - 2 of 3
  2.0 μM - 3 of 3
  2.5 μM - 1 of 3
  2.5 μM - 2 of 3
  2.5 μM - 3 of 3
  3.0 μM - 1 of 3
  3.0 μM - 2 of 3
  3.0 μM - 3 of 3
  5.0 μM - 1 of 3
  5.0 μM - 2 of 3
  5.0 μM - 3 of 3
```

TABLE 4
Mixture (same ratios, in equimolar concentrations)

| Amlodipine | Atorvastatin | sample |
|---|---|---|
| 1.0 μM | 1.0 μM | 1 of 3 |
| 1.0 μM | 1.0 μM | 2 of 3 |
| 1.0 μM | 1.0 μM | 3 of 3 |
| 2.0 μM | 2.0 μM | 1 of 3 |
| 2.0 μM | 2.0 μM | 2 of 3 |
| 2.0 μM | 2.0 μM | 3 of 3 |
| 2.5 μM | 2.5 μM | 1 of 3 |
| 2.5 μM | 2.5 μM | 2 of 3 |
| 2.5 μM | 2.5 μM | 3 of 3 |
| 3.0 μM | 3.0 μM | 1 of 3 |
| 3.0 μM | 3.0 μM | 2 of 3 |
| 3.0 μM | 3.0 μM | 3 of 3 |
| 5.0 μM | 5.0 μM | 1 of 3 |
| 5.0 μM | 5.0 μM | 2 of 3 |
| 5.0 μM | 5.0 μM | 3 of 3 |

The data were presented as mean ±SEM for each of the triplicate measurements. The data (calculation and plotting) were transferred to Microcal Origin Software (OriginLab Corp., Northampton, Mass.).

TABLE 5
NO Peak Measurements

| Substance Injected | NO Concentration, mean ± SEM |
|---|---|
| (concentration, μM) | (concentration, nM) |
| Amlodipine (0.25) | 24.21 ± 3.11 |
| Amlodipine (0.75) | 48.44 ± 5.83 |
| Amlodipine (1.00) | 53.50 ± 0.39 |
| Amlodipine (1.50) | 58.47 ± 11.00 |
| Amlodipine (2.00) | 72.25 ± 8.20 |
| Amlodipine (2.50) | 121.30 ± 24.11 |
| Amlodipine (3.00) | 151.26 ± 18.00 |
| Amlodipine (5.00) | 158.00 ± 19.81 |
| Atorvastatin (0.25) | 0.50 ± 0.02 |
| Atorvastatin (0.75) | 1.11 ± 0.12 |
| Atorvastatin (1.00) | 2.31 ± 0.53 |
| Atorvastatin (1.50) | 5.20 ± 1.21 |
| Atorvastatin (2.00) | 8.12 ± 3.10 |
| Atorvastatin (2.50) | 9.85 ± 3.00 |
| Atorvastatin (3.00) | 15.61 ± 2.19 |
| Atorvastatin (5.00) | 48.69 ± 2.48 |
| Amlodipine (5.00) + Atorvastatin (0.25) | 182.25 ± 21.14 |
| Amlodipine (5.00) + Atorvastatin (0.75) | 242.20 ± 24.00 |
| Amlodipine (5.00) + Atorvastatin (1.00) | 274.94 ± 22.06 |
| Amlodipine (5.00) + Atorvastatin (1.50) | 271.33 ± 15.20 |
| Amlodipine (5.00) + Atorvastatin (2.00) | 247.00 ± 6.11 |
| Amlodipine (5.00) + Atorvastatin (2.50) | 231.60 ± 7.80 |
| Amlodipine (5.00) + Atorvastatin (3.00) | 208.71 ± 30.74 |
| Amlodipine (5.00) + Atorvastatin (5.00) | 130.50 ± 15.12 |
| Amlodipine (1.00) + Atorvastatin (1.00) | 126 ± 18 |
| Amlodipine (2.00) + Atorvastatin (2.00) | 178 ± 7 |
| Amlodipine (2.50) + Atorvastatin (2.50) | 201 ± 11 |
| Amlodipine (3.00) + Atorvastatin (3.00) | 219 ± 6 |
| Amlodipine (5.00) + Atorvastatin (5.00) | 160 ± 71 |

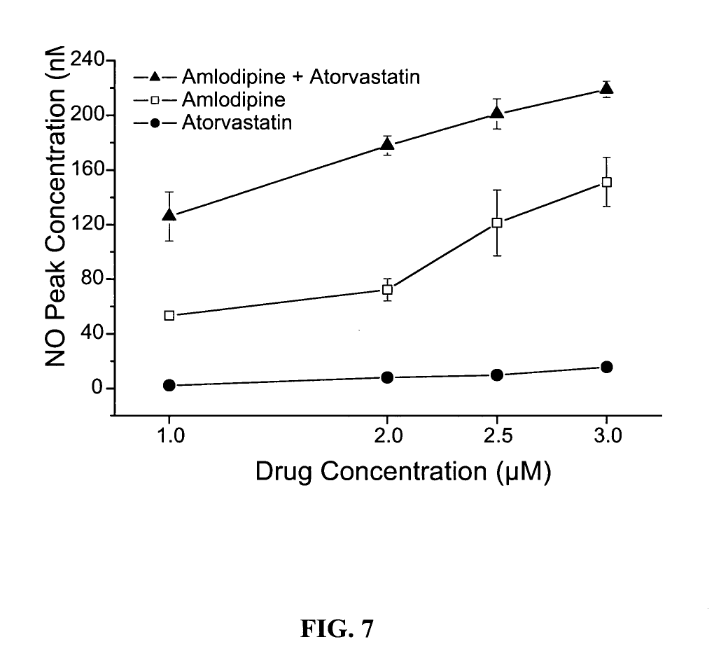

FIG. 7 depicts the separate and combined effects of amlodipine (open squares), atorvastatin (shaded circles), on NO release (nM) from human endothelial cells as a function of drug concentration (μM). At equimolar concentrations of amlodipine and atorvastatin, a pronounced synergistic effect was observed over a range of micromolar concentrations (1.0 through 3.0 μM). The release of NO was measured electrochemically with a sensitive porphyrinic sensor placed in close proximity to the cultured cell surface. The drug combination caused the release of NO from the human endothelial cells at levels that exceeded the expected additive effects of the drugs, and thus, indicated a clear synergistic effect.

It will now be apparent to those skilled in the art that other embodiments, improvements, details, and uses can be made that are consistent with the letter and spirit of the foregoing disclosure and within the scope of this patent and the appended claims.

1. A pharmaceutical composition for enhancing NO production comprising:
(a) a therapeutically effective amount of amlodipine;
(b) a therapeutically effective amount of an atorvastatin compound selected from the group consisting of atorvastatin and hydroxylated atorvastatin metabolite; and
(c) a therapeutically effective amount of one or more NO enhancing tertiary agents.

2. The pharmaceutical composition of claim 1 wherein amlodipine comprises a therapeutically effective derivative of amlodipine.

3. The pharmaceutical composition of claim 2 wherein the therapeutically effective derivative of amlodipine comprises amlodipine besylate.

4. The pharmaceutical composition of claim 1 wherein the atorvastatin compound comprises a therapeutically effective derivative of the atorvastatin compound.

5. The pharmaceutical composition of claim 4 wherein the therapeutically effective derivative of the atorvastatin compound is a hemicalcium salt.

6. The pharmaceutical composition of claim 1, wherein said NO enhancing tertiary agent is selected from the group consisting of L-arginine, tetrahydrobiopterin, ACE-inhibitor, antioxidant, β-blocker, angiotensin II type 1-receptor antagonist.

7. The pharmaceutical composition of claim 6, wherein said ACE-inhibitor is selected from the group consisting of ramipril, enalapril, quinapril, and alike.

8. The pharmaceutical composition of claim 6, wherein said antioxidant is selected from the group consisting of vitamin E, probucol, vitamin C, and alike.

9. The pharmaceutical composition of claim 6, wherein said β-blocker is selected from the group consisting of carvedilol, metoprolol, and alike.

10. The pharmaceutical composition of claim 6, wherein said angiotensin II type 1-receptor antagonist is selected from the group consisting of irbesartan, candesartan, valsartan, losartan, and alike.

11. The pharmaceutical composition of claim 1 wherein said pharmaceutical composition reduces the risk of arterial and related heart disease.

12. The pharmaceutical composition of claim 11, wherein said arterial and related heart disease is selected from the group consisting of hypertension, hyperlipdemia, atherosclerosis, arteriosclerosis, coronary artery disease, myocardial infarction, congestive heart failure, stroke, and angina pectoris.

13. A method of synergistically increasing nitric oxide production by endothelial cells comprising administering a therapeutically effective amount of a combination of amlodipine, an atorvastatin compound selected from the group consisting of atorvastatin and hydroxylated atorvastatin metabolite, and an NO enhancing tertiary agent.

14. A method of treating arterial and related heart disease comprising administering a therapeutically effective amount of a combination of amlodipine, an atorvastatin compound selected from the group consisting of atovastatin and hydroxylated atorvastatin metabolite, and an NO enhancing tertiary agent.
